(19) United States
(12) Patent Application Publication
(10) Pub. No.: US 2007/0270364 A1
(43) Pub. Date: Nov. 22, 2007

(54) Helicases

(75) Inventor: Ralf Janknecht, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/570,754

(22) PCT Filed: Jun. 10, 2005
(86) PCT No.: PCT/US05/20984

§ 371 Date: Jul. 23, 2007

Related U.S. Application Data
(60) Provisional application No. 60/579,830, filed on Jun. 15, 2004.

(51) Int. Cl. A61K 31/70; A61P 43/00; C12Q 1/68

(52) U.S. Cl. 514/44; 435/6

(57) ABSTRACT

This document provides methods and materials involved in diagnosing breast cancer in a mammal, identifying molecules that inhibit RNA helicase activity, and treating cancer (e.g., breast cancer).

### BACKGROUND

1. Technical Field

This document relates to methods and materials involved in diagnosing breast cancer in a mammal, identifying molecules that inhibit helicase (e.g., p68, p72, or p82 RNA helicase) activity, and treating cancer (e.g., breast cancer).

2. Background Information

RNA helicases form a large superfamily of conserved proteins that perform many essential functions, including RNA splicing, editing, nuclear export, translation, turnover, nonsense-mediated RNA decay, ribosome biogenesis, and RNA interference. Mechanistically, RNA helicases can act by unwinding duplex RNA, disrupting RNA:protein interactions or assisting in the correct folding of RNA [1,2]. In addition, RNA helicases may also be involved in gene transcription, for instance by stabilizing nascent transcripts or releasing completed transcripts from the template [3], and RNA helicases have indeed been shown to act as transcriptional cofactors [4-9].

Several reports demonstrated that RNA helicases can influence cell proliferation, DNA repair, and cell transformation [10-12]. The RNA helicase Rck/p54 is overexpressed in neuroblastoma, glioblastoma, rhabdomyosarcoma and lung cancer cells as well as in colorectal tumors [13,14]. Further, the DDX1 RNA helicase gene is often coamplified with N-myc in retinoblastomas and neuroblastomas and its coamplification correlates with a poorer prognosis [15-17].

p68 belongs to the DEAD box family of RNA helicases characterized by a conserved Walker B motif containing the sequence Asp-Glu-Ala-Asp (D-E-A-D) that is involved in ATP hydrolysis [18]. Several enzymatic activities have been ascribed to p68: (i) It hydrolyzes ATP. (ii) It is an RNA helicase that unwinds RNA. (iii) It has an RNA annealing activity, which together with the RNA helicase activity rearranges secondary RNA structures [19-22]. Accordingly, p68 is involved in RNA splicing and splice site selection [23,24]. In addition, studies on homologs of mammalian p68 RNA helicase have shown that p68 is required for RNA interference in Drosophila [25] and for proper cell growth, nonsense-mediated RNA decay, and rRNA processing in Saccharomyces cerevisiae [26-28].

Apart from its role in RNA metabolism, p68 RNA helicase acts in the regulation of gene transcription. It interacts with estrogen receptor-α (ER-α) and thereby stimulates ER-α dependent transcription [9]. Furthermore, p68 interacts with AIB1 (amplified in breast cancer 1), a steroid receptor coactivator overexpressed in the majority of all breast tumors [29], and with SRA (steroid receptor RNA activator), a cofactor that functions as an RNA [30]. It is thought that p68 RNA helicase, SRA and AIB1 synergistically stimulate ER-α dependent transcription [31]. In addition, p68 RNA helicase may affect transcription by interacting with RNA polymerase II and by stimulating the transcriptional coactivators CBP and p300 [32]. The homologous proteins CBP and p300 are endowed with acetyltransferase activity and thereby are capable of modulating chromatin structure as well as the function of a variety of different transcription factors [33,34].

### SUMMARY

This document involves methods and materials for diagnosing breast cancer in a mammal, identifying molecules that inhibit helicase (e.g., p68, p72, or p82 RNA helicase) activity, and treating cancer (e.g., breast cancer). The methods and materials provided herein can be used to diagnose mammals as having breast cancer. Diagnosing a mammal as having breast cancer cells can allow physicians to treat the mammal sooner than if the mammal was not diagnosed. Starting proper breast cancer treatments sooner can give the mammal a better chance of overcoming the breast cancer.

The methods and materials provided herein also can be used to identify molecules that inhibit helicase (e.g., p68, p72, or p82 RNA helicase) activity. Such molecules can be used to treat mammals having cancer cells (e.g., mammals having breast cancer, colorectal cancer, pancreatic cancer, brain cancer, or lung cancer). In addition, the methods and materials provided herein can be used to treat mammals having cancer cells (e.g., mammals having breast cancer, colorectal cancer, pancreatic cancer, brain cancer, or lung cancer) such that the number of cancer cells is reduced (e.g., a 10, 20, 30, 40, 50, 60, 70, 80, 90, or 100 percent reduction).

In general, this document features a method for determining whether or not a mammal has breast cancer. The method includes determining whether or not a sample from the mammal contains an elevated level of an RNA helicase (e.g., a p68, p72, or p82 RNA helicase) polypeptide, wherein the presence of the elevated level indicates that the mammal has the breast cancer. The mammal can be a human. The sample can be a breast tissue sample. The RNA helicase polypeptide can be a p68 RNA helicase polypeptide. The p68 RNA helicase polypeptide can contain the sequence set forth in SEQ ID NO:2. The RNA helicase polypeptide can be a p72 RNA helicase polypeptide. The p72 RNA helicase polypeptide can contain the sequence set forth in SEQ ID NO:14. The RNA helicase polypeptide can be a p82 RNA helicase polypeptide. The p82 RNA helicase polypeptide can contain the sequence set forth in SEQ ID NO:13.

In another aspect, this document features a method for treating a mammal having breast cancer. The method includes administering an inhibitor of an RNA helicase polypeptide activity to the mammal under conditions wherein the number of breast cancer cells within the mammal is reduced. The mammal can be a human. The inhibitor can reduce p68 RNA helicase polypeptide activity within breast cancer cells within the mammal. The inhibitor can be an siRNA molecule, anti-sense oligonucleotide, or ribozyme that reduces the expression level of a p68 RNA helicase polypeptide within the breast cancer cells. The inhibitor can reduce p72 RNA helicase polypeptide activity within breast cancer cells within the mammal. The inhibitor can be an siRNA molecule, anti-sense oligonucleotide, or ribozyme that reduces the expression level of a p72 RNA helicase polypeptide within the breast cancer cells. The inhibitor can reduce p82 RNA helicase polypeptide activity within breast cancer cells within the mammal. The inhibitor can be an siRNA molecule, anti-sense oligonucleotide, or ribozyme that reduces the expression level of a p82 RNA helicase polypeptide within the breast cancer cells.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1: Characterization of our anti-p68 antibody. (A) Anti-p68 Western blot of whole cell lysates from MDA-MB-231 and Hs578T human breast cancer cells. (B) Peptide competitions. Inclusion of a peptide encompassing p68 RNA helicase amino acids 555-576, but not of an unrelated peptide, suppressed recognition of endogenous p68 RNA helicase by anti-p68 antibody in Western blots of Hs578T cell extracts. (C) p68 RNA helicase expression in MDA-MB-231 or Hs578T breast cancer cells was visualized by immunostaining with anti-p68 antibody. DNA was stained with Hoechst dye.

FIG. 2: Immunohistochemical analysis of p68 expression (brown color) in human breast tissue (50x magnification). Cell nuclei were counterstained with light hematoxylin (blue color). (A) Invasive ductal carcinoma of the breast. (B) Normal breast.

FIG. 3: In vitro 14C-acetylation of various p68 RNA helicase amino acids fused to GST by the HAT domain of p300. The top shows a sketch of p68 with the eight conserved helicase domains. The left panel is an autoradiogram revealing acetylation of a GST-p68 polypeptide, whereas the right panel is a photograph of polypeptides stained with Coomassie Blue.

FIG. 4: In vivo acetylation of p68. (A) Extracts of 293T cells (non-transfected or transfected with p300) were challenged with control anti-GAL4 or anti-Acetyllysine (AcK) antibodies. Immunoprecipitates were subjected to Western blotting with anti-p68 antibodies. (B) Similarly, acetylated proteins were immunoprecitated from 293T cells transfected with the indicated combinations of p300, HER2/Neu and Myc-tagged p68. Then, p68 was detected by anti-Myc Western blotting.

FIG. 5: Co-immunoprecipitation of p68 and Miz-1. As indicated, HA-tagged p68 and Flag-tagged Miz-1 were coexpressed in 293T cells.

FIG. 6: Nucleic acid sequence that encodes a human p68 RNA helicase polypeptide (SEQ ID NO:1).

FIG. 7: Amino acid sequence of a human p68 RNA helicase polypeptide (SEQ ID NO:2).

FIG. 8: Characterization of an anti-p72 antibody. (A) Anti-p72 Western blot of whole cell lysates from human MDA-MB-468 breast cancer cells and human 293T transformed kidney cells. (B) Peptide competition experiments. Western blots of 293T cell extracts were simultaneously challenged with indicated peptides and anti-p72 antibody. The epitope peptide corresponds to amino acids 632-650 of human p72 RNA helicase (amino acids 711-729 of human p82 RNA helicase), against which an anti-p72 antibody was raised. (C) Immunohistochemical analysis of 68 different human breast tumor samples spotted onto microarrays. Staining was classified into three categories with regard to strength and also discriminated into nuclear and cytoplasmic staining.

FIG. 9: Nucleic acid sequence that encodes a human p82 and p72 RNA helicase polypeptide (SEQ ID NO:10). The coding sequence for a p82 isoform starts at nucleotide 75 and ends at nucleotide 2264 (SEQ ID NO:I 1), while the coding sequence for a p72 isoform starts at nucleotide 312 and ends at nucleotide 2264 (SEQ ID NO:12).

FIG. 10: Amino acid sequence of a human p82 and p72 RNA helicase polypeptide. The amino acid sequence for a p82 isoform starts at amino acid residue number 1 and ends at amino acid residue number 729 (SEQ ID NO:13), while the amino acid sequence for a p72 isoform starts at amino acid residue number 80 and ends at amino acid residue number 729 (SEQ ID NO: 14).

### DETAILED DESCRIPTION

This document provides methods and materials related to diagnosing breast cancer in a mammal (e.g., human, dog, cat, horse, cow, goat, pig, and rodent). For example, the invention provides methods and materials for determining whether or not a sample (e.g., breast tissue sample) from a mammal (e.g., a female human) contains an elevated level of an RNA helicase (e.g., p68, p72, or p82 RNA helicase) polypeptide. As disclosed herein, if the level of a p68, p72, or p82 RNA helicase polypeptide in a sample is an elevated level, then the mammal can be classified as having breast cancer. If the level of a p68, p72, or p82 RNA helicase polypeptide in a sample is not an elevated level, then the mammal can be classified as not having breast cancer.

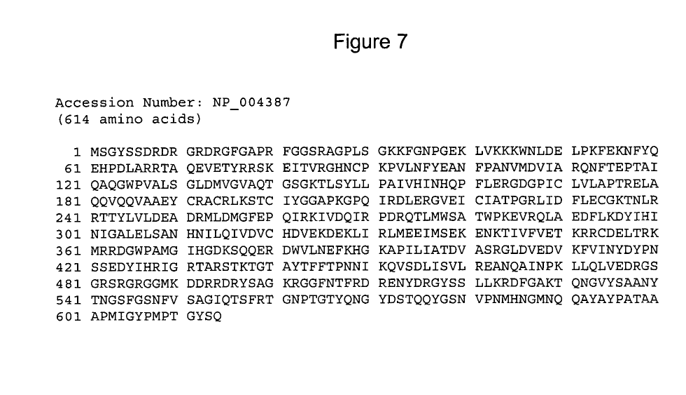

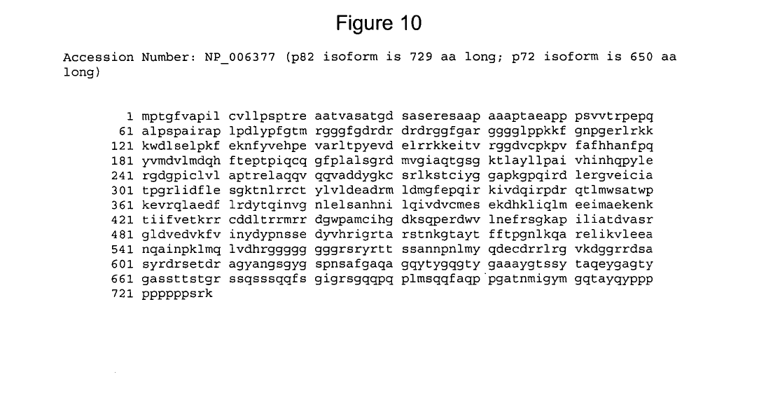

The level of a p68, p72, or p82 RNA helicase polypeptide can be determined by measuring any p68, p72, or p82 RNA helicase polypeptide including, without limitation, native and mutant p68, p72, or p82 RNA helicase polypeptides. Examples of p68 RNA helicase polypeptides include, without limitation, human p68 RNA helicase polypeptides (e.g., GenBank® accession number NP_{—}004387; FIG. 7), equine p68 RNA helicase polypeptides, canine p68 RNA helicase polypeptides, and mouse p68 RNA helicase polypeptides. Examples of p72 RNA helicase polypeptides include, without limitation, human p72 RNA helicase polypeptides (e.g., GenBank® accession number NP_{—}006377; FIG. 10), equine p72 RNA helicase polypeptides, canine p72 RNA helicase polypeptides, and mouse p72 RNA helicase polypeptides. Examples of p82 RNA helicase polypeptides include, without limitation, human p82 RNA helicase polypeptides (e.g., GenBank® accession number NP_{—}006377; FIG. 10), equine p82 RNA helicase polypeptides, canine p82 RNA helicase polypeptides, and mouse p82 RNA helicase polypeptides.

The term “elevated level” as used herein with respect to the level of an RNA helicase (e.g., a p68, p72, or p82 RNA helicase) polypeptide is any level that is greater than a reference level for an RNA helicase (e.g., a p68, p72, or p82 RNA helicase) polypeptide. The term “reference level” as used herein with respect to an RNA helicase (e.g., a p68, p72, or p82 RNA helicase) polypeptide is the level of an RNA helicase (e.g., a p68, p72, or p82 RNA helicase) polypeptide typically expressed by mammals free of cancer. For example, a reference level of a p68 RNA helicase polypeptide can be the average level of p68 RNA helicase polypeptide that is present in samples obtained from a random sampling of 50 healthy mammals. A reference level of a p72 RNA helicase polypeptide can be the average level of p72 RNA helicase polypeptide that is present in samples obtained from a random sampling of 50 healthy mammals.

It will be appreciated that levels from comparable samples are used when determining whether or not a particular level is an elevated level. For example, the average level of p68, p72, or p82 RNA helicase polypeptide present in breast tissue from a random sampling of mammals may be X units/g of breast tissue, while the average level of p68, p72, or p82 RNA helicase polypeptide present in lymph tissue from the breast region of the same random sampling of mammals may be Y units/g of lymph tissue. In this case, the reference level for p68, p72, or p82 RNA helicase polypeptide in breast tissue would be X units/g of breast tissue, and the reference level for p68, p72, or p82 RNA helicase polypeptide in lymph tissue would be Y units/g of lymph tissue. Thus, when determining whether or not the level of p6⁸, p72, or p82 RNA helicase polypeptide measured in breast tissue is elevated, the measured level would be compared to the reference level for p68, p72, or p82 RNA helicase polypeptide in breast tissue (i.e., X units/g of breast tissue).

An elevated level of an RNA helicase (e.g., a p68, p72, or p82 RNA helicase) polypeptide can be any level provided that the level is greater than a corresponding reference level for an RNA helicase (e.g., a p68, p72, or p82 RNA helicase) polypeptide. For example, an elevated level of a p68 RNA helicase polypeptide can be 0.5, 1, 2, 3, 4, 5, 6, 7, 8, 9, 10, 15, 20, or more times greater than the reference level for a p68 RNA helicase polypeptide. In addition, a reference level can be any amount. For example, a reference level for p68 RNA helicase polypeptide can be zero. In this case, any level of p68 RNA helicase polypeptide greater than zero would be an elevated level.

Any method can be used to determine the level of an RNA helicase (e.g., a p68, p72, or p82 RNA helicase) polypeptide present within a sample. For example, anti-p68 RNA helicase polypeptide antibodies can be used to determine the level of p68 RNA helicase polypeptide expression within a sample. In some embodiments, the level of a p68, p72, or p82 RNA helicase polypeptide present within a sample can be determined using polypeptide detection methods such as western blot and immunochemistry techniques. Another method that can be used to determine the level of a p68, p72, or p82 RNA helicase polypeptide present within a sample can be functional. For example, an ATPase assay can be used to determine whether or not a breast tissue sample contains an elevated level of a p68 RNA helicase polypeptide.

The level of an RNA helicase (e.g., a p68, p72, or p82 RNA helicase) polypeptide present within a sample also can be determined by measuring the level of an mRNA that encodes an RNA helicase (e.g., a p68, p72, or p82 RNA helicase) polypeptide. Any method can be used to measure the level of an RNA encoding an RNA helicase (e.g., a p68, p72, or p82 RNA helicase) polypeptide including, without limitation, PCR-based methods. For example, RT-PCR can be used with oligonucleotide primers designed to amplify nucleic acid (e.g., RNA) encoding a p68, p72, or p82 RNA helicase polypeptide. Any method can be used to identify primers capable of amplifying nucleic acid encoding a p68, p72, or p82 RNA helicase polypeptide. For example, a computer algorithm can be used to search a database (e.g., GenBank®) for p68 RNA helicase nucleic acid. Any method can be used to analyze the amplified products. For example, amplified products corresponding to p68, p72, or p82 RNA helicase mRNA can be separated by gel electrophoresis, and the level of p68, p72, or p82 RNA helicase-specific product determined by densiotometry. Alternatively, the level of p68, p72, or p82 RNA helicase-specific product can be determined by quantitative RT-PCR using fluorescent beacons or dyes.

Any type of sample can be used to evaluate the level of an RNA helicase (e.g., a p68, p72, or p82 RNA helicase) polypeptide including, without limitation, breast tissue or lymphatic tissue from the breast region. In addition, any method can be used to obtain a sample. For example, a breast tissue sample can be obtained by a tissue biopsy. Once obtained, a sample can be manipulated prior to measuring the level of an RNA helicase (e.g., a p68, p72, or p82 RNA helicase) polypeptide. For example, a breast tissue sample can be treated such that total mRNA is obtained. Once obtained, the total mRNA can be evaluated to determine the level of p68, p72, or p82 RNA helicase mRNA present. In another example, a breast tissue sample can be disrupted to obtain a cell lysate. Once obtained, the cell lysate can be analyzed using anti-RNA helicase polypeptide antibodies (e.g., anti-p68, -p72, or -p82 RNA helicase polypeptide antibodies) to determine the level of RNA helicase polypeptide (e.g., p68, p72, or p82 RNA helicase polypeptide) present within the sample.

This document also provides methods and materials to assist medical or research professionals in determining whether or not a mammal has breast cancer. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of an RNA helicase (e.g., a p68, p72, or p82 RNA helicase) polypeptide in a sample, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document provides methods and materials related to identifying molecules that inhibit RNA helicase activity (e.g., p68, p72, or p82 RNA helicase activity). For example, this document provides methods and materials for identifying inhibitors of a p68, p72, or p82 RNA helicase activity such as transcriptional activation and/or ATPase activity. Such inhibitors can be identified using a luciferase (or other detectable marker) transcriptional activation assay or an ATPase assay. In some embodiments, inhibitors can be identified using a soft agar assay that assesses colony formation and/or cell growth.

This document provides methods and materials related to treating mammals having cancer cells (e.g., mammals having breast cancer, colorectal cancer, pancreatic cancer, brain cancer, or lung cancer). For example, the methods and materials provided herein can be used to treat mammals having cancer cells (e.g., mammals having breast cancer, colorectal cancer, pancreatic cancer, brain cancer, or lung cancer) such that the number of cancer cells is reduced (e.g., a 10, 20, 30, 40, 50, 60, 70, 80, 90, or 100 percent reduction). In particular, a compound is administer to a mammal having cancer cells such that the compound reduces the level of an RNA helicase (e.g., a p68, p72, or p82 RNA helicase) polypeptide activity within the mammal's cancer cells. Any type of compound can be used to reduce the level of an RNA helicase (e.g., a p68, p72, or p82 RNA helicase) polypeptide activity within a mammal's cancer cells. For example, any inhibitor of a p68, p72, or p82 RNA helicase polypeptide activity (e.g., ATPase inhibitors) can be used. In some embodiments, antisense nucleic acid molecules, siRNA molecules, RNAi constructs, and/or PNA oligos can be used to reduce the level of p68, p72, or p82 RNA helicase polypeptide activity within a mammal's cancer cells by reducing the level of p68, p72, or p82 RNA helicase polypeptide expression within the cells.

In some cases, one or more compounds can be administered to a mammal having cancer cells such that the level of more than one RNA helicase polypeptide activity within the mammal's cancer cells is reduced. For example, a single compound can be administered to reduce the level of p68 and p72 RNA helicase polypeptide activity. In another example, two compounds can be administered to a mammal having cancer cells: one compound (e.g., an siRNA molecule targeting expression of a p68 RNA helicase polypeptide) that can reduce the level of a p68 RNA helicase polypeptide activity, and another compound (e.g., an siRNA molecule targeting expression of both p72 and p82 RNA helicase polypeptides) that can reduce the level of both p72 and p82 RNA helicase polypeptide activity.

The invention will be further described in the following examples, which do not limit the scope of the invention described in the claims.

### EXAMPLES

### Example 1

### Expression of p68 Polypeptide in Breast Cancer Cells

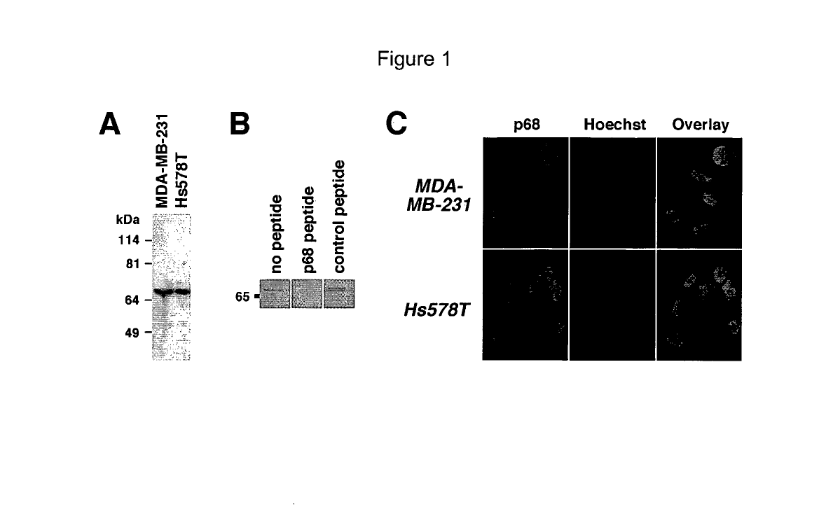

An anti-p68 polypeptide antibody was developed to immunohisto-chemically stain tissue samples from breast cancer patients for p68 RNA helicase and to molecularly study this protein. This antibody is directed against amino acid residues 555-576 of human p68 RNA helicase and has been affinity-purified. In a Western blot, this antibody recognized a single polypeptide of ˜68 kDa in two different cell lines (FIG. 1A). To demonstrate that this polypeptide is indeed a p68 RNA helicase polypeptide, peptide competition experiments were performed. A polypeptide that included amino acid residues 555-576 of p68 RNA helicase polypeptide efficiently suppressed antibody recognition of the 68 kDa polypeptide, while an unrelated polypeptide did not (FIG. 1B), indicating that the 68 kDa polypeptide recognized by the antibody was a p68 RNA helicase polypeptide.

Similarly, the anti-p68 polypeptide antibody specifically recognized endogenous p68 RNA helicase in immunostainings [32]. In two representative human breast cancer cell lines, MDA-MB-231 and Hs578T, p68 RNA helicase polypeptide was mainly present in the cell nucleus (consistent with its role as a transcriptional cofactor), but cytoplasmic staining to various degrees was additionally observable (FIG. 1C).

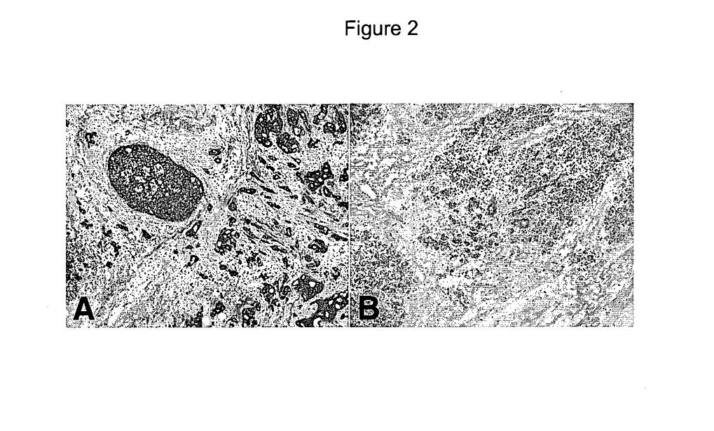

The expression of p68 RNA helicase polypeptide in breast tissue was examined. Ten specimens of breast tissue were obtained from patients diagnosed with breast cancer. In nine of these breast tumor specimens, overexpression of a p68 RNA helicase polypeptide was observed when compared to normal controls (FIG. 2), suggesting that p68 RNA helicase polypeptide overexpression is involved in breast tumor development.

A systematic, unbiased analysis of breast tumor microarrays representing 64 different human breast tumors confirmed the initial results that p68 was over-expressed in a significant proportion of human breast tumors (Table 1). Briefly, tissue micro arrays were stained with a p68 antibody, thereby analyzing 64 different human breast tumors. A pathologist graded the staining in the cell nucleus and cytoplasm of the breast tumor specimens as being strong, medium, or little/none.

TABLE 1
p68 staining in human breast tumor.

| | Nucleus | Cytoplasm |
|---|---|---|
| strong: | 9 | 13 |
| medium: | 15 | 15 |
| little/none: | 40 | 36 |

Posttranslational modification of proteins can be an important measure to modulate their function. Acetylation, in particular by CBP and p300, can be a process that regulates oncoproteins and tumor suppressors [33,34]. In order to understand a protein's mode of action, one can determine how posttranslational modifications such as acetylation affect its function. In particular, enhanced polypeptide levels of p68 RNA helicase in these tumors may be a consequence of altered posttranslational modification.

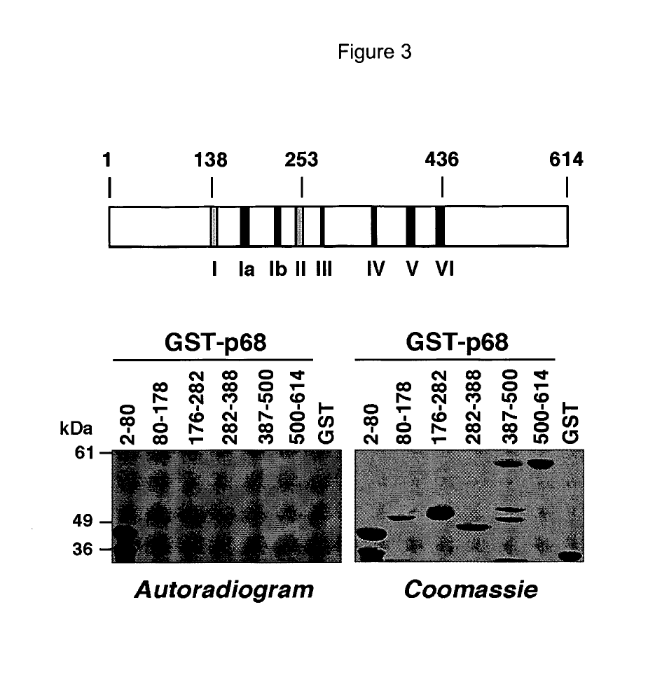

p68 RNA helicase polypeptide interacts with the coactivators and acetyltransferases CBP and p300 [32]. One question is whether p68 RNA helicase polypeptide not only interacts with but is also acetylated by CBP and p300. To this end, experiments were performed to determine whether GST-p68 fusion polypeptides were acetylated by the histone acetyltransferase (HAT) region of p300 in vitro. p300 indeed acetylated p68 polypeptides exclusively within its N-terminal 80 amino acids (FIG. 3).

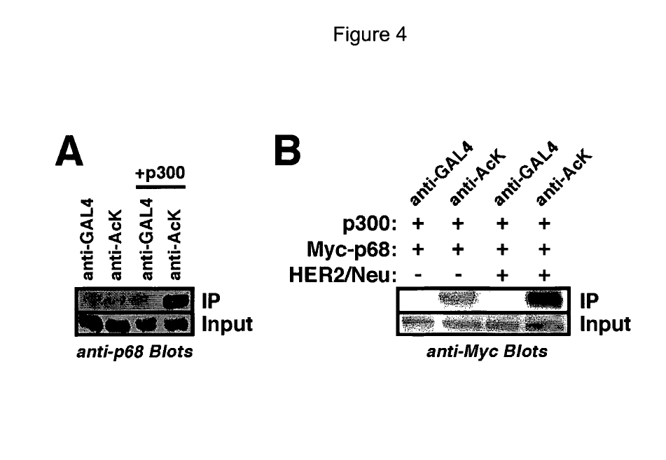

Experiments were performed to determine whether p68 RNA helicase polypeptides are also acetylated on a lysine residue(s) in vivo. Here, antibodies that recognize acetylated lysine residues was used. One such antibody (anti-AcK) and a control anti-GAL4 antibody were utilized for immunoprecipitation. After SDS-PAGE, any immunoprecipitated (acetylated) p68 RNA helicase polypeptide was detected by anti-p68 Western blotting. The results indicated that endogenous p68 RNA helicase polypeptide is, to a low extent, acetylated in 293T cells. Importantly, upon p300 overexpression, the level of acetylation of endogenous p68 RNA helicase polypeptide increased (FIG. 4A). Then, whether acetylation of p68 RNA helicase might be affected by HER2/Neu, a proto-oncoprotein that is overexpressed in 30% of human breast tumors correlating with an adverse prognosis, was also tested [40,41]. In vivo acetylation of p68 polypeptide was enhanced by HER2/Neu (FIG. 4B), probably due to the fact that HER2/Neu overexpression leads to an activation of the acetyltransferase activity of p300 [42]. In conclusion, p68 RNA helicase polypeptide is acetylated in vivo, most likely by p300 in particular upon HER2/Neu stimulation.

### Example 2

### Impact of Acetylation on p68 RNA Helicase Function

p68 RNA helicase polypeptides are acetylated by p300 within its N-terminal 80 amino acids. A consensus site for p300 acetylation is a lysine residue that is flanked by a positively charged amino acid at either position -3 or +4 or both [43]. Inspection of p68 amino acids 1-80 revealed four such lysine residues at positions 40, 43, 56, and 80.

These lysine residues are mutated to arginine, which can prevent acetylation but is otherwise a conserved exchange of one basic amino acid for another. First, one can test whether mutated GST-p68(2-80) molecules are still subject to in vitro acetylation by p300. If more than one lysine residue affects the level of acetylation, one can produce combination mutants in order to block acetylation completely.

The next step is the introduction of respective acetylation site mutations into full-length p68 RNA helicase polypeptides. One can assess whether these K→R mutations indeed suppress in vivo acetylation of p68 (in the presence and absence of coexpressed p300) by employing anti-acetyllysine antibodies as shown in FIG. 4. If so, this would indicate that the respective lysine residues are acetylated in vivo, most likely by p300.

In addition, the remaining five lysine residues within amino acids 1-80 of p68 RNA helicase are mutated and investigated for their ability to become acetylated in vitro and in vivo.

Having mapped all acetylation sites, their importance in p68-dependent transcription is assessed. As reported before [32], the TPA oncogene responsive unit (TORU) is synergistically activated by p68 RNA helicase and p300. p68 acetylation site mutants can differ from wild-type p68 in their ability to activate transcription, demonstrating that acetylation is an important regulator of p68 RNA helicase activity. In addition, one can assess how acetylation, probably by preventing ubiquitylation at the same lysine residues, may increase the half-life of p68 RNA helicase and thereby cause the observed overexpression of p68 RNA helicase in breast tumors.

One also can assess how acetylation of p68 RNA helicase affects its cooperation with ER-α, its enzymatic activities (RNA helicase and ATPase), its intracellular localization, and its ability to transform cells. In addition, acetylation-specific anti-p68 antibodies can be made and used to stain human breast tumor specimens. HER2/Neu polypeptide overexpression can correlate with p68 acetylation status.

### Example 3

### Interaction of Miz-1 with p68 RNA Helicase

One of the first oncogenes shown to be amplified in ˜20% of all human breast tumors has been c-Myc, and c-Myc polypeptide overexpression was later confirmed in >50% of all breast tumors [44,45]. Furthermore, Myc amplification correlates with a poor prognosis, and mice expressing Myc under the control of the MMTV (mouse mammary tumor virus) promoter/enhancer develop mammary tumors. As such, c-Myc is an important player in breast tumor formation.

Myc polypeptides exert their tumorigenic action through both up- and downregulation of genes. For instance, Myc activates the cdk4 and cyclin D2 genes and, on the other hand, represses p21CIP1 and p15INK4b, two cell cycle inhibitors [46,47]. Recently, it has become obvious that Myc-mediated repression primarily occurs through the recruitment of Myc by Miz-1 (Myc-interacting Zn finger protein-1) that can bind to the initiator region of gene promoters [48]. Miz-1 normally activates transcription of the p21CIP1 and p15INK4b genes, but Myc association with Miz-1 suppresses this activating function on gene transcription which is, at least in part, due to preventing the interaction between Miz-1 and p300 [49-54].

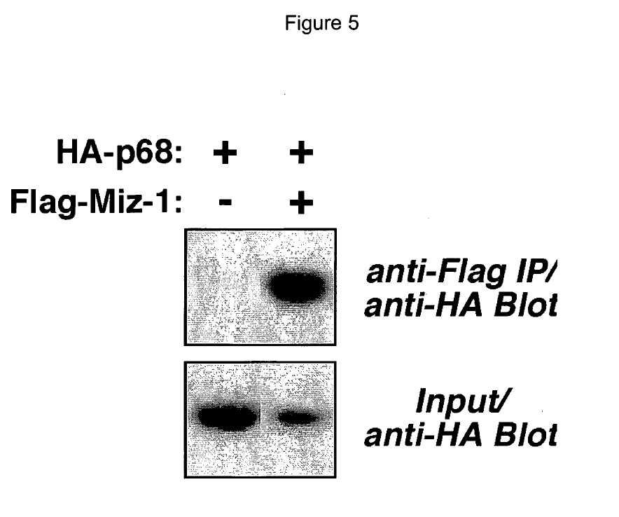

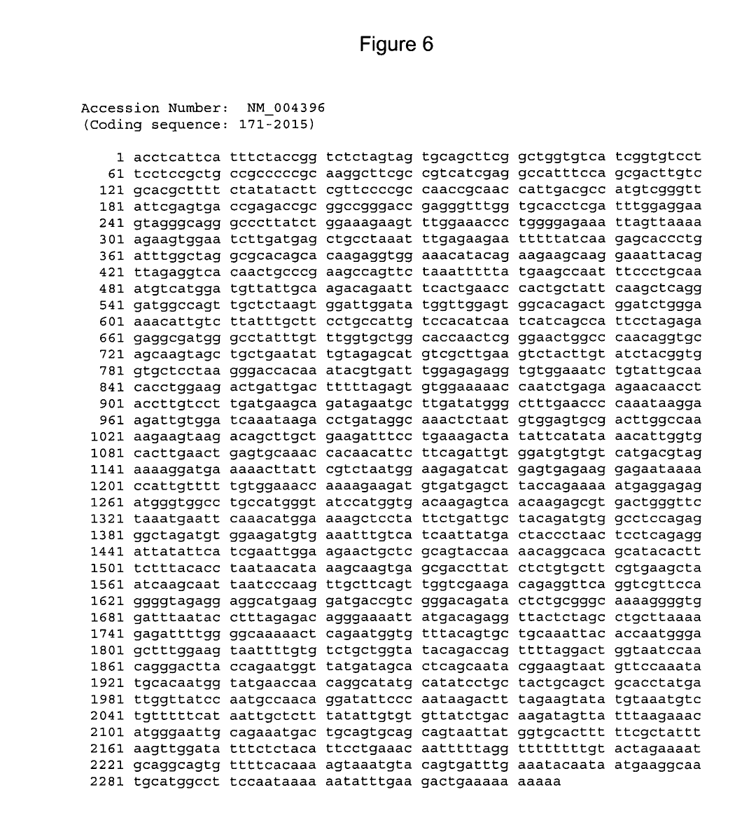

Miz-1 was found to be a potential p68 interaction partner in a yeast two-hybrid screen. As shown in FIG. 5, p68 RNA helicase and Miz-1 also interact in mammalian cells.

The functional consequences of the association of p68 RNA helicase with Miz-1 is studied. p68 RNA helicase, as does c-Myc, may suppress gene activation mediated by Miz-1. In order to test this, two experiments are performed. In the first one, Miz-1 polypeptide is overexpressed with and without p68 RNA helicase, and activation of the endogenous p21CIP1 and p15INK4b genes as well as of the respective gene promoters cloned in front of luciferase utilizing various breast cancer cell lines is observed by RT-PCR. The p68 RNA helicase polypeptide may reduce Miz-1-dependent gene activation. Since p68 RNA helicase is highly expressed in breast cancer cell lines (see e.g. FIG. 1), additional overexpression of p68 may not be effective and thus the experimental outcome inconclusive. Therefore, in a second experiment, one can knock-down p68 RNA helicase polypeptide expression by siRNA. This can result in the enhancement of Miz-1-dependent activation of the p21CIP1 and p15INK4b genes. An alternative hypothesis to be pursued is that p68 RNA helicase, instead of repressing Miz-1 on its own, augments Myc in its ability to repress Miz-1. Altogether, these experiments will point out a mechanism of how p68 RNA helicase overexpression promotes breast tumorigenesis by blocking the expression of p21CIP1 and p15NK4b.

### Example 4

### Generation of MMTV-p68 Transgenic Mice

p68 RNA helicase polypeptide is overexpressed in breast tumors. To investigate the potential of p68 RNA helicase polypeptide in tumorigenesis, a transgenic mouse model that overexpresses p68 RNA helicase polypeptide in mammary tissue is made. To this end, the MMTV promoter/enhancer, which has often been used to drive expression of oncogenes in mammary tissue leading to breast tumor formation [55], is used.

First, it will be determined if MMTV-p68 mice develop spontaneously breast tumors in contrast to normal mice. If so, that will demonstrate that p68 RNA helicase polypeptide is capable of eliciting breast cancer. If not, then additional transgenic mice can be made to determine whether two or more oncogenes are needed. For example, transgenic mice can be made by crossing MMTV-p68 mice to MMTV-HER2/Neu mice (of course, other mice like MMTV-Myc or MMTV-Cyclin D1 would also be suitable, but HER2/Neu may aggravate p68 overexpression by inducing its acetylation). It is possible that p68 polypeptide overexpression can lead to a more severe breast tumor phenotype in MMTV-HER2/Neu mice. Alternatively, MMTV-p68 mice can be mated with p53±mice that are known to be tumor prone [56]; for instance, p53±mice in the BALB/c strain develop breast tumors in 55% of all females [57]. Since p53−/−mice generally succumb to lymphomas within six month due to a severe tumor phenotype [56], such mice may not be very suitable to observe long term modifying effects of other genetic alterations on tumorigenesis. It is possible that p68 overexpression collaborates with the loss of the tumor suppressor p53 in the development of breast tumors. Similarly, MMTV-p72 and MMTV-p82 mice can be generated and utilized.

In summary, the results presented herein demonstrate that p68 RNA helicase polypeptides are involved in breast cancer.

### Example 5

### Role of p68 RNA Helicase Polypeptides in Tumor Cells

The following experiments are performed to investigate the impact of p68 RNA helicase polypeptides on tumor cell phenotype. Utilizing RNA interference, p68 RNA helicase polypeptide expression is suppressed in human breast tumor cell lines, and the resulting changes in growth rate and anchorage-independent growth are assessed. Briefly, siRNA molecules are useful agents to assess the effects of downregulating p68 RNA helicase polypeptide expression in cells. In particular, if p68 is a proto-oncogene, then its downregulation can obstruct cell transformation. Established breast cancer cell lines are transfected with siRNA molecules that target RNA interference of p68, and the resultant effect on growth in soft agar or cell proliferation is measured. The following human p68 RNA helicase cDNA sequences can be used as siRNA target sequences:

| | |
|---|---|
| 1. TAAGGAAGATTGTGGATCA | (SEQ ID NO:3) |
| 2. TAAGACCTGATAGGCAAAC | (SEQ ID NO:4) |
| 3. ACCACAACATTCTTCAGAT | (SEQ ID NO:5) |
| 4. ACTTATTCGTCTAATGGAA | (SEQ ID NO:6) |
| 5. AGAAGATGTGATGAGCTTA | (SEQ ID NO:7) |
| 6. GACAGAGGTTCAGGTCGTT | (SEQ ID NO:8) |
| 7. GAACTGCTCGCAGTACCAA | (SEQ ID NO:9) |

In addition, soft-agar and colony formation assays are used to assess the ability of p68 polypeptides to promote β-catenin-mediated transformation of NIH3T3 and rat RK3E cells. These studies can define whether p68 RNA helicase polypeptides are involved in the maintenance and/or induction of cell transformation.

Similar techniques can be used to reduce expression of p72 or p82 RNA helicases. For example, the following human p72 and p82 RNA helicase cDNA sequences can be used as siRNA target sequences:

| | |
|---|---|
| 1. GAGACGCTGTGATGATCTG | (SEQ ID NO:15) |
| 2. GATGTCAAGTTTGTGATCA | (SEQ ID NO:16) |

### Example 6

### Interaction Between p68 RNA Helicase Polypeptides and β-Catenin Polypeptides

p68 RNA helicase polypeptides were found to stimulate β-catenin-mediated gene transcription. The following experiments are performed to determine the mechanism by which p68 RNA helicase polypeptides activate β-catenin function. Coimmuno-precipitation assays are used to examine the forms of β-catenin and p68 RNA helicase polypeptides and interactions. In addition, experiments are performed to determine whether p68 RNA helicase polypeptide overexpression leads to accumulation of β-catenin in the nucleus, a prerequisite for its oncogenic action. This can unravel how p68 polypeptides directly stimulate the nuclear functions of β-catenin.

Similar experiments are performed to determine the mechanism by which p72 and p82 RNA helicase polypeptides activate β-catenin function.

### Example 7

### p68 RNA Helicase Polypeptide Overexpression

The following experiments are performed to determine the mechanism underlying p68 polypeptide overexpression in tumors. First, transcriptional upregulation of the p68 RNA helicase gene is examined using the Breast Cancer Profiling Array (Clonetech; 30 matched normal and tumor specimens) to assess p68 mRNA levels. Second, protein stabilization by post-translational modification is assessed. Briefly, p68 polypeptides are isolated from breast cancer cells and normal control cells with anti-p68 antibodies. The purified p68 polypeptides are subjected to mass-spectrometric analysis. Mass differences can reveal the exact kind of post-translational modification(s) that distinguish between p68 from cancer and normal cells. These studies are fundamental to future studies determining how the p68 gene promoter is upregulated (e.g., by ER-α) or how p68 polypeptide becomes stabilized by post-translational modification (e.g., by blocking ubiquitylation sites via acetylation or sumoylation of the same lysine residues).

Similar experiments are performed to determine the mechanism underlying p72 and p82 polypeptide overexpression in tumors.

### Example 8

### Identifying Inhibitors of p68 RNA Helicase Polypeptide Function Via Luciferase Activity

p68 RNA helicase polypeptides can activate the TORU (TPA oncogene responsive unit) luciferase construct in CV-1 cells (Rossow and Janknecht, Oncogene 22: 151-6 (2003)). In addition, p68 RNA helicase polypeptides can augment transcriptional activation of an estrogen-dependent promoter construct, ERE-tk-luc, in 293 cells (Endoh et al., Mol. Cell. Biol., 19:5363-72 (1999)). Either of these two reporter gene readouts are used to screen the effect of chemical compounds on the activity of p68 RNA helicase polypeptides. Briefly, cells transfected with a reporter construct and nucleic acid encoding a p68 RNA helicase polypeptide are compared to cells transfected with the reporter construct and control empty vector. In case of the ERE-tk-luc reporter construct, cells are additionally treated with 10-8 M 17β-estradiol after transfection. Specifically, cells are transfected using lipofectamine 2000 (Invitrogen) according to the manufacturer's instruction in 10 mm dishes. Twelve hours after transfection, the cells are treated with a chemical compound (test substance) or vehicle. Twenty-four hours later, the cells are lysed in 250 βL of 25 mM Tris, 2 mM EDTA, 10% glycerol, 1% Triton-X100, and 2 mM DTT (pH 7.8). After a clear-spin, 50 μL of the supernatant are mixed with 300 μL of 25 mM glycylglycine, 15 mM MgSO₄, and 5 mM ATP. Then, luciferase activity is measured by chemiluminescence in a Berthold Lumat after injection of 100 μL of 0.25 mM Luciferin.

Test substances that reduce luciferase activity in these assay systems compared to respective vehicle controls are potential inhibitors of p68 RNA helicase polypeptide activity.

Similar procedures are performed using p72 or p82 polypeptides to identify potential inhibitors of p72 or p82 RNA helicase polypeptide activity.

### Example 9

### Identifying Inhibitors of p68 RNA Helicase Polypeptide Function Via ATPase Activity

Six cm dishes of 293T cells are transfected with 4.5 μg 6Myc-p68 RNA helicase expression vector. Twelve hours after transfection, the cells are treated with test substance or control vehicle. After another 24 hours, immunoprecipitations are performed. Briefly, cells are lysed in 600 μL of 10 mM Tris, 50 mM NaCl, 50 mM NaF, 1% Triton X-100, 0.2 mM DTT, and a protease inhibitor cocktail, (pH 7.1). After a clearspin, the supernatant is challenged with anti-Myc (9E10) monoclonal antibodies and beads consisting of protein A coupled to sepharose. Beads are spun down after 2 hours and washed 3 times in lysis buffer and 2 times in ATPase buffer (20 mM Hepes (pH 7.4), 50 mM NaCl, 5 mM MgCl₂, and 1 mM DTT). Finally, beads are resuspended in 100 μL ATPase buffer.

15 μL of this suspension plus 0.1 mM ATP plus 1 μCi γ-³²P-ATP is incubated at 37° C. for 30 minutes, and the reaction then stopped with 5 μL of EDTA. The conversion of ATP into ADP and Pi is monitored by chromatography on PEI Cellulose F TLC plates (MERCK 1.05725). To this end, 0.5 μL of the reaction mixture is spotted onto the TLC plate, which is developed in 0.5 M LiCl/1 M formic acid. After 60 minutes, plates are dryed, wrapped in Saran-wrap, and exposed to film. The appearance of radioactive Pi is the readout for the ATPase activity of p68 RNA helicase. A test substance that reduces ATPase activity as compared to a vehicle control can be an inhibitor of p68 RNA helicase polypeptide activity.

Similar procedures are performed using p72 and p82 polypeptides to identify potential inhibitors of p72 or p82 RNA helicase polypeptide activity.

### Example 10

### Identifying Inhibitors of p68 RNA Helicase Polypeptide Function Via a Soft Agar Assay

NIH3T3 cells are transfected with nucleic acid encoding a p68 RNA helicase polypeptide using lipofectamine 2000 (Invitrogen) according to the manufacturer's protocol in a 100 mm dish. 24 hours after transfection, 50,000 transfected cells are seeded in DMEM/10% fetal calf serum/0.33% agar (Agar Noble, Difco) and plated on top of a hardened complete medium/0.5% agar base in a 6-well plate. Both agar components are included for the test substance and the control vehicle. Cells are fed every 5-7 days with 3 mL of complete medium/0.33% agar (plus test substance or control vehicle) and incubated until colonies become readily visible (14-28 days). Experiments are performed in triplicate and repeated at least twice. Test substances that reduce the number of colonies compared to vehicle are potential inhibitors of p68 RNA helicase polypeptide function.

Similar procedures are performed using p72 or p82 polypeptides to identify potential inhibitors of p72 or p82 RNA helicase polypeptide activity.

### Example 11

### Expression of p72 Polypeptide in Breast Cancer Cells

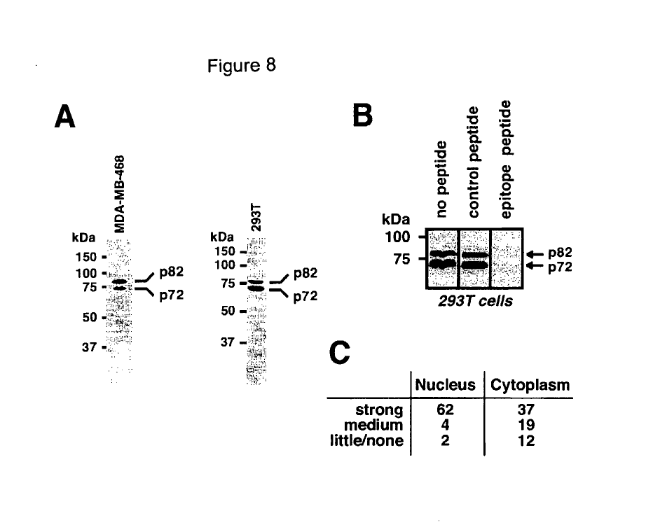

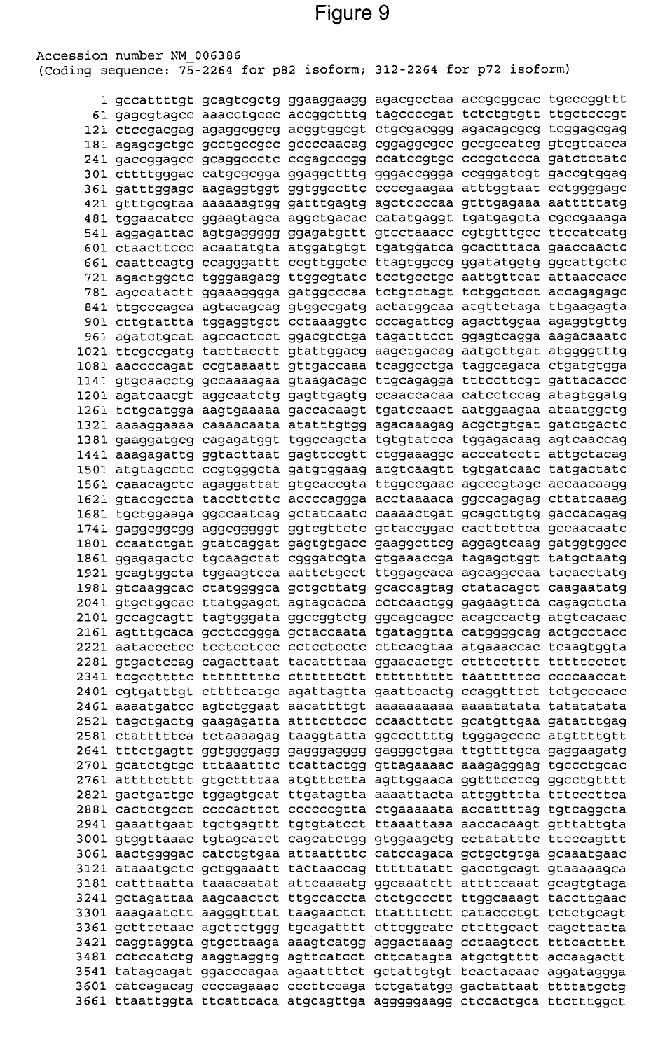

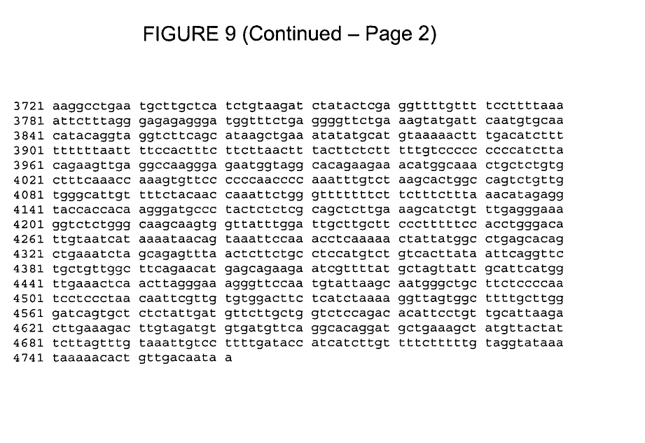

An anti-p72 polypeptide antibody was developed to immunohisto-chemically stain tissue samples from breast cancer patients for p72 RNA helicase and to molecularly study this protein. This antibody is directed against amino acid residues 632-650 of human p72 RNA helicase (amino acid residues 711-729 of human p82 RNA helicase) and has been affinity-purified. This sequence was GQTAYQYPPPPPPPPPSRK (SEQ ID NO: 17). In a Western blot, this antibody recognized a two polypeptides, one about 72 kDa and the other about 82 kDa in two different cell lines (FIG. 8A).

To demonstrate that these polypeptides are indeed p72 and p82 RNA helicase polypeptides, peptide competition experiments were performed. A polypeptide that included amino acid residues 632-650 of human p72 RNA helicase polypeptide efficiently suppressed antibody recognition of the 72 kDa and 82 kDa polypeptides, while an unrelated polypeptide did not (FIG. 8B), indicating that the 72 kDa polypeptide recognized by the antibody was a p72 RNA helicase polypeptide and that the 82 kDa polypeptide recognized by the antibody was a p82 RNA helicase polypeptide.

A systematic, unbiased analysis of breast tumor microarrays representing 68 different human breast tumors confirmed that p72 and p82 RNA helicase polypeptides were over-expressed in a significant proportion of human breast tumors (FIG. 8C). Briefly, tissue micro arrays were stained with a p72 antibody, thereby analyzing 68 different human breast tumors. A pathologist graded the staining in the cell nucleus and cytoplasm of the breast tumor specimens as being strong, medium, or little/none.

### REFERENCES

1. Tanner, N. K. and Linder, P. (2001) DExD/H box RNA helicases: from generic motors to specific dissociation functions. Mol. Cell 8, 251-262.
2. Silverman, E., Edwalds-Gilbert, G. and Lin, R. J. (2003) DExD/H-box proteins and their partners: helping RNA helicases unwind. Gene 312, 1-16.
3. Eisen, A. and Lucchesi, J. C. (1998) Unraveling the role of helicases in transcription. Bioessays 20, 634-641.
4. Nakajima, T., Uchida, C., Anderson, S. F., Lee, C. G., Hurwitz, J., Parvin, J. D. and Montminy, M. (1997) RNA helicase A mediates association of CBP with RNA polymerase II. Cell 90, 1107-1112.
5. Yan, X., Mouillet, J. F., Ou, Q. and Sadovsky, Y. (2003) A novel domain within the DEAD-box protein DP 103 is essential for transcriptional repression and helicase activity. Mol. Cell. Biol. 23, 414-423.
6. Rajendran, R. R., Nye, A. C., Frasor, J., Balsara, R. D., Martini, P. G. and Katzenellenbogen, B. S. (2003) Regulation of nuclear receptor transcriptional activity by a novel DEAD box RNA helicase (DP97). J. Biol. Chem. 278, 4628-4638.
7. Westermarck, J., Weiss, C., Saffrich, R., Kast, J., Musti, A. M., Wessely, M., Ansorge, W., Seraphin, B., Wilm, M., Valdez, B. C. and Bohmann, D. (2002) The DEXD/H-box RNA helicase RHII/Gu is a co-factor for c-Jun-activated transcription. EMBO J. 21, 451-460.
8. Aratani, S., Fujii, R., Oishi, T., Fujita, H., Amano, T., Ohshima, T., Hagiwara, M., Fukamizu, A. and Nakajima, T. (2001) Dual roles of RNA helicase A in CREB-dependent transcription. Mol. Cell. Biol. 21, 4460-4469.
9. Endoh, H., Maruyama, K., Masuhiro, Y., Kobayashi, Y., Goto, M., Tai, H., Yanagisawa, J., Metzger, D., Hashimoto, S. and Kato, S. (1999) Purification and identification of p68 RNA helicase acting as a transcriptional coactivator specific for the activation function 1 of human estrogen receptor alpha. Mol. Cell. Biol. 19, 5363-5372.
10. Wood, M. A., McMahon, S. B. and Cole, M. D. (2000) An ATPase/helicase complex is an essential cofactor for oncogenic transformation by c-Myc. Mol. Cell 5, 321-330.
11. Cantor, S. B., Bell, D. W., Ganesan, S., Kass, E. M., Drapkin, R., Grossman, S., Wahrer, D. C., Sgroi, D. C., Lane, W. S., Haber, D. A. and Livingston, D. M. (2001) BACH1, a novel helicase-like protein, interacts directly with BRCA1 and contributes to its DNA repair function. Cell 105, 149-160.
12. Liu, Z. P. and Olson, E. N. (2002) Suppression of proliferation and cardiomyocyte hypertrophy by CHAMP, a cardiac-specific RNA helicase. Proc. Natl. Acad. Sci. USA 99, 2043-2048.
13. Akao, Y., Marukawa, O., Morikawa, H., Nakao, K., Kamei, M., Hachiya, T. and Tsujimoto, Y. (1995) The rck/p54 candidate proto-oncogene product is a 54-kilodalton D-E-A-D box protein differentially expressed in human and mouse tissues. Cancer Res. 55, 3444-3449.
14. Nakagawa, Y., Morikawa, H., Hirata, I., Shiozaki, M., Matsumoto, A., Maemura, K., Nishikawa, T., Niki, M., Tanigawa, N., Ikegami, M., Katsu, K. and Akao, Y. (1999) Overexpression of rck/p54, a DEAD box protein, in human colorectal tumours. Br. J. Cancer 80, 914-917.
15. Godbout, R. and Squire, J. (1993) Amplification of a DEAD box protein gene in retinoblastoma cell lines. Proc. Natl. Acad. Sci. USA 90, 7578-7582.
16. Squire, J. A., Thomer, P. S., Weitzman, S., Maggi, J. D., Dirks, P., Doyle, J., Hale, M. and Godbout, R. (1995) Co-amplification of MYCN and a DEAD box gene (DDX1) in primary neuroblastoma. Oncogene 10, 1417-1422.
17. George, R. E., Kenyon, R. M., McGuckin, A. G., Malcolm, A. J., Pearson, A. D. and Lunec, J. (1996) Investigation of co-amplification of the candidate genes ornithine decarboxylase, ribonucleotide reductase, syndecan-1 and a DEAD box gene, DDX1, with N-myc in neuroblastoma. United Kingdom Children's Cancer Study Group. Oncogene 12, 1583-1587.
18. Caruthers, J. M. and McKay, D. B. (2002) Helicase structure and mechanism. Curr. Opin. Struct. Biol. 12, 123-133.
19. Hirling, H., Scheffner, M., Restle, T. and Stahl, H. (1989) RNA helicase activity associated with the human p68 protein. Nature 339, 562-564.
20. Iggo, R. D. and Lane, D. P. (1989) Nuclear protein p68 is an RNA-dependent ATPase. EMBO J. 8, 1827-1831.
21. Rossler, O. G., Straka, A. and Stahl, H. (2001) Rearrangement of structured RNA via branch migration structures catalysed by the highly related DEAD-box proteins p68 and p72. Nucleic Acids Res. 29, 2088-2096.
22. Huang, Y. and Liu, Z. R. (2002) The ATPase, RNA unwinding, and RNA binding activities of recombinant p68 RNA helicase. J. Biol. Chem. 277, 12810-12815.
23. Liu, Z. R. (2002) p68 RNA helicase is an essential human splicing factor that acts at the U1 snRNA-5′ splice site duplex. Mol. Cell. Biol. 22, 5443-5450.
24. Guil, S., Gattoni, R., Carrascal, M., Abian, J., Stevenin, J. and Bach-Elias, M. (2003) Roles of hnRNP A1, SR proteins, and p68 helicase in c-H-ras alternative splicing regulation. Mol. Cell. Biol. 23, 2927-2941.
25. Ishizuka, A., Siomi, M. C. and Siomi, H. (2002) A Drosophila fragile X protein interacts with components of RNAi and ribosomal proteins. Genes Dev. 16, 2497-2508.
26. Iggo, R. D., Jamieson, D. J., MacNeill, S. A., Southgate, J., McPheat, J. and Lane, D. P. (1991) p68 RNA helicase: identification of a nucleolar form and cloning of related genes containing a conserved intron in yeasts. Mol. Cell. Biol. 11, 1326-1333.
27. Barta, I. and Iggo, R. (1995) Autoregulation of expression of the yeast Dbp2p ‘DEAD-box’ protein is mediated by sequences in the conserved DBP2 intron. EMBO J. 14, 3800-3808.
28. Bond, A. T., Mangus, D. A., He, F. and Jacobson, A. (2001) Absence of Dbp2p alters both nonsense-mediated mRNA decay and rRNA processing. Mol. Cell. Biol. 21, 7366-7379.
29. Anzick, S. L., Kononen, J., Walker, R. L., Azorsa, D. O., Tanner, M. M., Guan, X. Y., Sauter, G., Kallioniemi, O. P., Trent, J. M. and Meltzer, P. S. (1997) AIB1, a steroid receptor coactivator amplified in breast and ovarian cancer. Science 277, 965-968.
30. Lanz, R. B., McKenna, N. J., Onate, S. A., Albrecht, U., Wong, J., Tsai, S. Y., Tsai, M. J. and O'Malley, B. W. (1999) A steroid receptor coactivator, SRA, functions as an RNA and is present in an SRC-1 complex. Cell 97, 17-27.
31. Watanabe, M., Yanagisawa, J., Kitagawa, H., Takeyama, K., Ogawa, S., Arao, Y., Suzawa, M., Kobayashi, Y., Yano, T., Yoshikawa, H., Masuhiro, Y. and Kato, S. (2001) A subfamily of RNA-binding DEAD-box proteins acts as an estrogen receptor alpha coactivator through the N-terminal activation domain (AF-1) with an RNA coactivator, SRA. EMBO J. 20, 1341-1352.
32. Rossow, K. L. and Janknecht, R. (2003) Synergism between p68 RNA helicase and the transcriptional coactivators CBP and p300. Oncogene 22, 151 -156.
33. Goodman, R. H. and Smolik, S. (2000) CBP/p300 in cell growth, transformation, and development. Genes Dev. 14, 1553-1577.
34. Janknecht, R. (2002) The versatile functions of the transcriptional coactivators p300 and CBP and their roles in disease. Histol. Histopathol. 17, 657-668.
35. Causevic, M., Hislop, R. G., Kemohan, N. M., Carey, F. A., Kay, R. A., Steele, R. J. and Fuller-Pace, F. V. (2001) Overexpression and poly-ubiquitylation of the DEAD-box RNA helicase p68 in colorectal tumours. Oncogene 20, 7734-7743.
36. Wei, Y. and Hu, M. H. (2001) [The study of P68 RNA helicase on cell transformation]. Yi Chuan Xue Bao 28, 991-996.
37. Anderson, E. (2002) The role of oestrogen and progesterone receptors in human mammary development and tumorigenesis. Breast Cancer Res. 4, 197-201.
38. Keen, J. C. and Davidson, N. E. (2003) The biology of breast carcinoma. Cancer 97, 825-833.
39. Jemal, A., Murray, T., Samuels, A., Ghafoor, A., Ward, E. and Thun, M. J. (2003) Cancer statistics, 2003. CA Cancer J. Clin. 53, 5-26.
40. Hynes, N. E. and Stem, D. F. (1994) The biology of erbB-2/neu/HER-2 and its role in cancer. Biochim. Biophys. Acta 1198, 165-184.
41. Holbro, T., Civenni, G. and Hynes, N. E. (2003) The ErbB receptors and their role in cancer progression. Exp. Cell Res. 284, 99-110.
42. Goel, A. and Janknecht, R. (2003) Acetylation-Mediated Transcriptional Activation of the ETS Protein ER81 by p300, P/CAF, and HER2/Neu. Mol. Cell. Biol. 23, 6243-6254.
43. Thompson, P. R., Kurooka, H., Nakatani, Y. and Cole, P. A. (2001) Transcriptional coactivator protein p300. Kinetic characterization of its histone acetyltransferase activity. J. Biol. Chem. 276, 33721-33729.
44. Hynes, N. E. and Lane, H. A. (2001) Myc and mammary cancer: Myc is a downstream effector of the ErbB2 receptor tyrosine kinase. J. Mammary Gland Biol. Neoplasia 6, 141-150.
45. Liao, D. J. and Dickson, R. B. (2000) c-Myc in breast cancer. Endocr. Relat. Cancer 7, 143-164.
46. Lüscher, B. (2001) Function and regulation of the transcription factors of the Myc/Max/Mad network. Gene 277, 1-14.
47. Gartel, A. L. and Shchors, K. (2003) Mechanisms of c-myc-mediated transcriptional repression of growth arrest genes. Exp. Cell Res. 283, 17-21.
48. Peukert, K., Staller, P., Schneider, A., Carmichael, G., Hanel, F. and Eilers, M. (1997) An alternative pathway for gene regulation by Myc. EMBO J. 16, 5672-5686.
49. Staller, P., Peukert, K., Kiermaier, A., Seoane, J., Lukas, J., Karsunky, H., Moroy, T., Bartek, J., Massague, J., Hanel, F. and Eilers, M. (2001) Repression of p15INK4b expression by Myc through association with Miz-1. Nat. Cell Biol. 3, 392-399.
50. Seoane, J., Pouponnot, C., Staller, P., Schader, M., Eilers, M. and Massague, J. (2001) TGFbeta influences Myc, Miz-1 and Smad to control the CDK inhibitor p15INK4b. Nat. Cell Biol. 3, 400-408.
51. Herold, S., Wanzel, M., Beuger, V., Frohme, C., Beul, D., Hillukkala, T., Syvaoja, J., Saluz, H. P., Haenel, F. and Eilers, M. (2002) Negative regulation of the mammalian UV response by Myc through association with Miz-1. Mol. Cell 10, 509-521.
52. Seoane, J., Le, H. V. and Massague, J. (2002) Myc suppression of the p21(Cip1) Cdk inhibitor influences the outcome of the p53 response to DNA damage. Nature 419, 729-734.
53. van de Wetering, M., Sancho, E., Verweij, C., de Lau, W., Oving, I., Hurlstone, A., van der Horn, K., Batlle, E., Coudreuse, D., Haramis, A. P., Tjon-Pon-Fong, M., Moerer, P., van den Born, M., Soete, G., Pals, S., Eilers, M., Medema, R. and Clevers, H. (2002) The beta-catenin/TCF-4 complex imposes a crypt progenitor phenotype on colorectal cancer cells. Cell 111, 241-250.
54. Wu, S., Cetinkaya, C., Munoz-Alonso, M. J., von der Lehr, N., Bahram, F., Beuger, V., Eilers, M., Leon, J. and Larsson, L. G. (2003) Myc represses differentiation-induced p21CIP1 expression via Miz-1-dependent interaction with the p21 core promoter. Oncogene 22, 351-360.
55. Hutchinson, J. N. and Muller, W. J. (2000) Transgenic mouse models of human breast cancer. Oncogene 19, 6130-6137.
56. Blackburn, A. C. and Jerry, D. J. (2002) Knockout and transgenic mice of Trp53: what have we learned about p53 in breast cancer? Breast Cancer Res. 4, 101-111.
57. Kuperwasser, C., Hurlbut, G. D., Kittrell, F. S., Dickinson, E. S., Laucirica, R., Medina, D., Naber, S. P. and Jerry, D. J. (2000) Development of spontaneous mammary tumors in BALB/c p53 heterozygous mice. A model for Li-Fraumeni syndrome. Am. J. Pathol. 157, 2151-2159.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether or not a mammal has breast cancer, said method comprising determining whether or not a sample from said mammal contains an elevated level of an RNA helicase polypeptide, wherein the presence of said elevated level indicates that said mammal has said breast cancer.

2. The method of claim 1, wherein said mammal is a human.

3. The method of claim 1, wherein said sample is a breast tissue sample.

4. The method of claim 1, wherein said RNA helicase polypeptide is a p68 RNA helicase polypeptide.

5. The method of claim 4, wherein said p68 RNA helicase polypeptide comprises the sequence set forth in SEQ ID NO:2.

6. The method of claim 1, wherein said RNA helicase polypeptide is a p72 RNA helicase polypeptide.

7. The method of claim 4, wherein said p72 RNA helicase polypeptide comprises the sequence set forth in SEQ ID NO:14.

8. The method of claim 1, wherein said RNA helicase polypeptide is a p82 RNA helicase polypeptide.

9. The method of claim 4, wherein said p82 RNA helicase polypeptide comprises the sequence set forth in SEQ ID NO:13.

10. A method for treating a mammal having breast cancer, said method comprising administering an inhibitor of an RNA helicase polypeptide activity to said mammal under conditions wherein the number of breast cancer cells within said mammal is reduced.

11. The method of claim 10, wherein said mammal is a human.

12. The method of claim 10, wherein said inhibitor reduces p68 RNA helicase polypeptide activity within breast cancer cells within said mammal.

13. The method of claim 12, wherein said inhibitor is an siRNA molecule that reduces the expression level of a p68 RNA helicase polypeptide within said breast cancer cells.

14. The method of claim 10, wherein said inhibitor reduces p72 RNA helicase polypeptide activity within breast cancer cells within said mammal.

15. The method of claim 14, wherein said inhibitor is an siRNA molecule that reduces the expression level of a p72 RNA helicase polypeptide within said breast cancer cells.

16. The method of claim 10, wherein said inhibitor reduces p82 RNA helicase polypeptide activity within breast cancer cells within said mammal.

17. The method of claim 16, wherein said inhibitor is an siRNA molecule that reduces the expression level of a p82 RNA helicase polypeptide within said breast cancer cells.
